(19) United States
(12) Patent Application Publication
(10) Pub. No.: US 2024/0024141 A1
(43) Pub. Date: Jan. 25, 2024

(54) APPARATUSES FOR STENT DELIVERY AND POSITIONING FOR TRANSLUMINAL APPLICATION

(71) Applicant: Covidien LP, Mansfield, MA (US)

(72) Inventors: Kevin M. Morris, Santa Rosa, CA (US); Siddhi K. Desai, Santa Clara, CA (US); Kee Sein Lee, West Sacramento, CA (US); Mark L. Stiger, Santa Rosa, CA (US)

(21) Appl. No.: 18/479,931

(22) Filed: Oct. 3, 2023

Related U.S. Application Data
(63) Continuation-in-part of application No. 17/581,660, filed on Jan. 21, 2022.

(51) Int. Cl. A61F 2/966; A61B 18/14; A61L 29/14; A61L 29/04

(52) U.S. Cl. CPC A61F 2/966; A61B 18/1492; A61L 29/14; A61L 29/041; A61B 2018/00535

(57) ABSTRACT

A system for delivering a stent into a body lumen includes an inner tubular member configured to advance through an access site in a wall of a body lumen for delivering a stent into the body lumen and an electrode configured to create the access site in the wall of the body lumen. A tubular sleeve is disposed coaxially over the distal end portion of the inner tubular member and is configured to thermally insulate at least a portion of the inner tubular member.

### CROSS-REFERENCE TO RELATED APPLICATIONS

This application is a continuation-in-part of U.S. patent application Ser. No. 17/581,660, filed on Jan. 21, 2022.

### BACKGROUND

Diseases and disorders of the gallbladder, pancreas, and bile ducts (i.e., pancreaticobiliary system) are associated with significant morbidity, mortality, and impaired quality of life. Obstructions, tumors, injuries, leakages, inflammation, infection, and lesions can occur in these structures, which can eventually lead to conditions such as biliary colic, cholecystitis, choledocholithiasis, cholelithiasis, pancreatitis, pancreatic duct stone formations, and chronic abdominal pain. Diseases of the pancreaticobiliary system may also be associated with nutritional disorders, such as malnutrition, obesity, and high cholesterol.

To treat a biliary obstruction, a clinician may perform a stent delivery procedure to place a stent across the body lumen to bypass the obstruction. In general, a stent delivery procedure may include placing an endoscope into the gastrointestinal tract and accessing the bile duct with a catheter (e.g., a fine needle aspiration (FNA) catheter). A guidewire may then be deployed through the catheter and into the bile duct. Once the guidewire is in place, a catheter (e.g., a FNA catheter) may be withdrawn and a stent or other treatment device may be advanced over the guidewire into the bile duct. After the stent is placed in the bile duct, the clinician may withdraw the stent delivery system. Alternatively, a stent delivery system may initially access the bile duct to provide access for the guidewire to be advanced into the bile duct prior to further advancement of the stent delivery system into the bile duct. After advancement of the guidewire into the bile duct, the stent delivery system is then further advanced over the guidewire and into the bile duct. Another alternative includes a stent delivery system accessing the bile duct without the use of a guide wire.

### SUMMARY

In accordance with an aspect of the disclosure, a system for delivering a stent into a body lumen includes an inner tubular member configured to advance through an access site in a wall of a body lumen and a stent configured to be disposed coaxially on the inner tubular member. The system also includes an outer sheath disposed coaxially along at least a portion of the inner tubular member such that the stent is disposed between the inner tubular member and the outer sheath while the stent is in an undeployed configuration. The system also includes a distal cutting element and a tubular sleeve. The distal cutting element is coupled with a distal end portion of the inner tubular member and has an electrode configured to create the access site in the wall of the body lumen. The tubular sleeve is disposed coaxially over a distal end portion of the inner tubular member and is configured to thermally insulate at least a portion of the inner tubular member.

In some aspects of the present disclosure, the tubular sleeve is formed of PTFE.

In some aspects of the present disclosure, the tubular sleeve is formed of silicone, ceramic, or a ceramic-impregnated material.

In some aspects of the present disclosure, a distal end of the tubular sleeve is disposed distal to a distal edge of the electrode.

In some aspects of the present disclosure, a distal end of the tubular sleeve is flush with a distal edge of the electrode.

In some aspects of the present disclosure, a distal end of the sleeve is disposed proximal to a distal edge of the electrode.

In some aspects of the present disclosure, the tubular sleeve overlaps the distal end portion of the inner tubular member by about 6 mm.

In some aspects of the present disclosure, a distal end of the inner tubular member is spaced proximally from a distal end of the electrode.

In some aspects of the present disclosure, an anchoring component is disposed at the distal end portion of the inner tubular member and is configured to retain a distal portion of the stent in place along the inner tubular member as the outer sheath is retracted proximally to deploy the stent. Upon retraction of the outer sheath, the stent releases from the anchoring component and expands into a deployed configuration within the body lumen.

In some aspects of the present disclosure, a proximal marker is disposed around the inner tubular member and positioned such that a proximal end of the stent abuts against the proximal marker while the stent is in the undeployed configuration. The proximal marker is configured to indicate a location of the proximal end of the stent.

In some aspects of the present disclosure, a middle member is disposed around the inner tubular member proximal to the proximal marker such that a proximal end of the proximal marker abuts a distal end of the middle member.

In some aspects of the present disclosure, the distal cutting element includes a cover having an extension extending distally from a distal end of the cover, and the electrode is looped around the extension.

In some aspects of the present disclosure, the cover is formed of a material selected from the group consisting of silicone, ceramic, and PTFE.

Another system for delivering a stent into a body lumen provided in accordance with the present disclosure includes an inner tubular member configured to advance through an access site in a wall of a body lumen for delivering a stent into the body lumen, an outer sheath disposed coaxially along at least a portion of the inner tubular member, a distal cutting element, and a tubular sleeve. The distal cutting element is coupled with a distal end portion of the inner tubular member and has an electrode configured to create the access site in the wall of the body lumen. The tubular sleeve is disposed coaxially over the distal end portion of the inner tubular member and is configured to thermally insulate at least a portion of the inner tubular member. A distal end of the tubular sleeve is disposed distal to a distal edge of the electrode.

In some aspects of the present disclosure, the tubular sleeve is formed of PTFE.

In some aspects of the present disclosure, the tubular sleeve is formed of silicone, ceramic, or a ceramic-impregnated material.

In some aspects of the present disclosure, the tubular sleeve overlaps the distal end portion of the inner tubular member by about 6 mm.

In some aspects of the present disclosure a distal end of the inner tubular member is spaced proximally from a distal end of the electrode.

In some aspects of the present disclosure, the distal cutting element includes a cover having an extension extending distally from a distal end of the cover, and the electrode is looped around the extension.

Another system for delivering a stent into a body lumen provided in accordance with the present disclosure includes an inner tubular member formed of PEEK, an electrode coupled with a distal end portion of the inner tubular member, and a tubular sleeve formed of PTFE, silicone, ceramic, or a ceramic-impregnated material. The inner tubular member is configured to advance through an access site in a wall of a body lumen for delivering a stent into the body lumen. The electrode is coupled with a distal end portion of the inner tubular member and is configured to create the access site in the wall of the body lumen. The tubular sleeve is disposed coaxially over the distal end portion of the inner tubular member and is configured to thermally insulate at least a portion of the inner tubular member.

Certain embodiments of the present disclosure may include some, all, or none of the above advantages or features. One or more other technical advantages or features may be readily apparent to those skilled in the art from the figures, descriptions, and claims included herein. Moreover, while specific advantages or features have been enumerated above, various embodiments may include all, some, or none of the enumerated advantages or features.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a system for providing access to a body lumen in accordance with aspects of the present disclosure.

FIG. 2 illustrates a system for providing access to a body lumen in accordance with aspects of the present disclosure.

FIG. 3A illustrates a cross-sectional view of an anchoring component in accordance with aspects of the present disclosure.

FIG. 3B illustrates a perspective view of an anchoring component in accordance with aspects of the present disclosure.

FIG. 4 illustrates a distal cutting element with a coiled electrode wire in accordance with aspects of the present disclosure.

FIG. 5 illustrates a distal cutting element with single electrode wires in accordance with aspects of the present disclosure.

FIG. 6 illustrates a distal cutting element with a spiral electrode wire in accordance with aspects of the present disclosure.

FIG. 7 illustrates a distal cutting element with an electrode tube in accordance with aspects of the present disclosure.

FIG. 8 illustrates a stent in accordance with aspects of the present disclosure.

FIG. 9A illustrates a stent delivery system with a flared portion of the stent deployed in accordance with aspects of the present disclosure.

FIG. 9B illustrates a stent delivery system with the stent fully deployed in accordance with aspects of the present disclosure.

FIG. 10 illustrates a schematic view of an inner tubular member disposed within a tubular sleeve in accordance with aspects of the present disclosure.

FIG. 11 illustrates an embodiment of a distal cutting element with an embedded electrode wire in accordance with aspects of the present disclosure.

### DETAILED DESCRIPTION

The present disclosure is generally directed to delivering a stent and positioning the stent for transluminal application. In certain procedures described herein, to place a stent within a body lumen, the luminal wall is pierced, and a stent delivery system is advanced through the hole (i.e., access site or access hole) and positioned at the target site to bypass an obstruction. The stent is then deployed from the stent delivery system, and the stent delivery system is withdrawn back out of the lumen through the same hole. If the stent is not accurately and precisely deployed, the stent may be unable to form a bridge between two body lumens and therefore, may be unable to bypass the obstruction. For example, if the distal portion and/or proximal portion of the stent is deployed short of (e.g., below or out of) the access hole, the stent may be unable to connect the two body lumens and unable to form an alternate route to bypass the obstruction. In some cases, inaccurate deployment may result in fluid from the lumen leaking out into the surrounding tissue and organs, which may potentially cause serious discomfort or other medical complications.

In some cases, the stent may be positioned through an access site in a wall of a first body lumen (through a wall of a second body lumen) after using a distal cutting element to create the access site. In some cases, a catheter (e.g. a FNA catheter) may be used to create an access site in a wall of a first body lumen (through a wall of a second body lumen). A guidewire may then be deployed through the catheter and into the first body lumen. Once the guidewire is in place, a catheter (e.g. a FNA catheter) may be withdrawn and a stent may be positioned (over the guide wire) through the access site in the wall of the first body lumen. Alternatively, the stent may be positioned through an access site in a wall of a first body lumen (through a wall of a second body lumen) using a distal cutting element to create the access site, then a guide wire may be advanced into the first body lumen before further advancing the stent into the first body lumen. The stent may be in an undeployed configuration such that the stent is housed within the outer sheath. The system for delivering the stent may include an inner tubular member that is configured to advance through an access site in a wall of the body lumen. The stent may be disposed coaxially onto the inner tubular member. The outer sheath may be disposed coaxially along at least a portion of the inner tubular member such that the stent is disposed between the inner tubular member and the outer sheath while the stent is in an undeployed configuration.

The system for delivering the stent may further include an anchoring component disposed at a distal portion of the inner tubular member. In some cases, a distal portion of the stent may be disposed coaxially along the anchoring component such that the distal portion of the stent is disposed between the anchoring component and the outer sheath while the stent is in the undeployed configuration. The anchoring component may be configured to retain a distal portion of the stent in place along the inner tubular member as the outer sheath is retracted proximally to deploy the stent. For example, the outer sheath may be retracted proximally and past the anchoring component disposed at a distal portion of an inner tubular member while the anchoring component and the inner tubular memory remain stationary.

Upon retraction of the outer sheath, the stent releases from the anchoring component and expands into a deployed configuration within the body lumen. For example, the distal portion of the stent may deploy from the outer sheath into a deployed configuration within the first body lumen based on retracting the outer sheath past the anchoring component. In such cases, the distal portion of the stent may accurately and precisely deploy in the first body lumen, thereby enabling the stent to be able to form a bridge between two body lumens and bypass the obstruction. For example, the distal portion of the stent may deploy in the first body lumen and anchor itself within the first body lumen.

The stent may be an example of a non-foreshortening stent. For example, the stent may include a helical wrapping pattern that may be configured to reduce a foreshortening of the stent body upon deployment from the undeployed configuration to the deployed configuration to less than ten percent of a length of the stent body in the undeployed configuration. In such cases, the stent may remain in place during retraction of the outer sheath and deployment of the distal portion as the outer sheath continues to be retracted proximally. The non-foreshortening stent may enable accurate deployment by positioning the stent within the body lumen and maintaining the position of the stent within the body lumen before and after deploying the stent.

The system for delivering the stent may further include a proximal marker disposed around the inner tubular member and positioned such that a proximal end of the stent abuts against the proximal marker while the stent is in the undeployed configuration. The proximal marker may be configured to indicate a location of the proximal end of the stent within the system endoscopically and/or fluoroscopically. For example, a distal end of the marker may be aligned with (typically some distance away from) a wall of the second body lumen to ensure that the proximal portion of the stent precisely deploys within the second body lumen and allows the stent to bridge between two body lumens upon expansion. The outer sheath may be withdrawn past the distal end of the marker to expand the proximal portion of the stent. In such cases, the proximal portion of the stent may expand from within the outer sheath such that upon fully exiting the outer sheath, the proximal portion expands to a deployed configuration within the second body lumen. The deployed configuration may be an example of the stent fully exiting the outer sheath and expanding between the first body lumen and the second body lumen, thereby providing an alternative route to bypass the obstruction.

In some cases, the stent may include a stent body having a first diameter and a first length in a deployed configuration. The stent may include a helical wrapping pattern that is at least partially covered with a material. In some cases, the stent may include at least two anchoring members coupled with a distal portion and a proximal portion, respectively, of the stent body. For example, the stent may include a first anchoring member coupled with a distal portion of the stent body and configured to increase a diameter of the distal portion of the stent body to a second diameter greater than the first diameter. The stent may further include a second anchoring member coupled with a proximal portion of the stent body and configured to increase a diameter of the proximal portion of the stent body to the second diameter greater than the first diameter. Each of the anchoring members may be configured to anchor the distal and proximal portions of the stent within the respective body lumens such that the stent remains in a fixed position.

Embodiments of the present disclosure are now described in detail with reference to the drawings. As used herein, the term “clinician” refers to a doctor, surgeon, nurse, or any other care provider and may include support personnel. The term “proximal” will refer to the portion of the device or component thereof that is closer to the clinician and the term ‘distal” will refer to the portion of the device or component thereof that is farther from the clinician.

Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent.

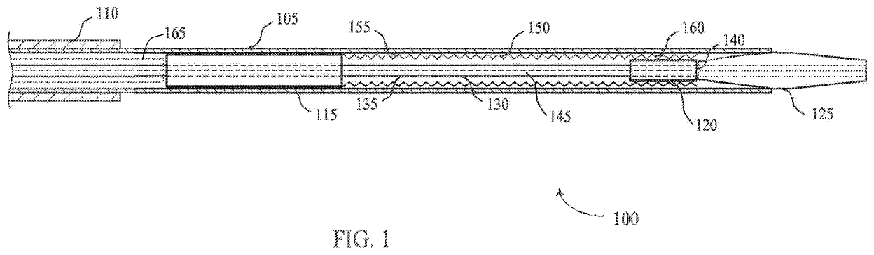

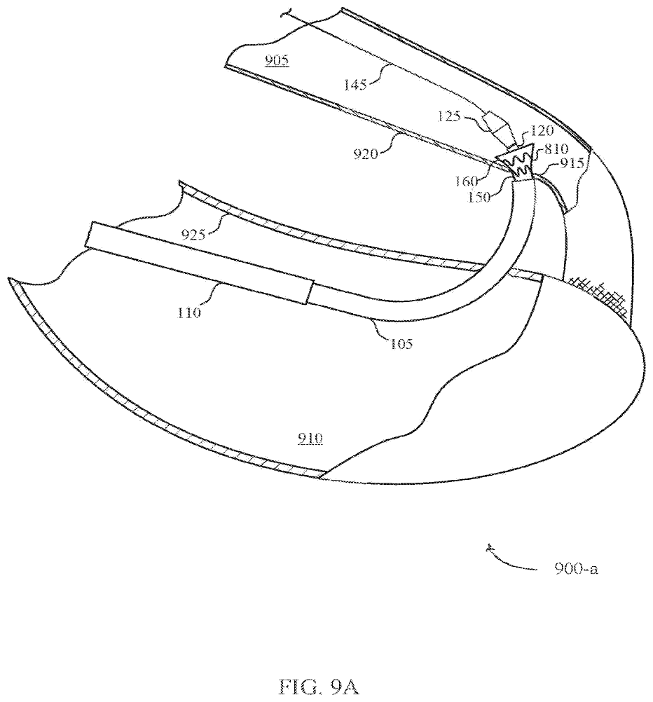

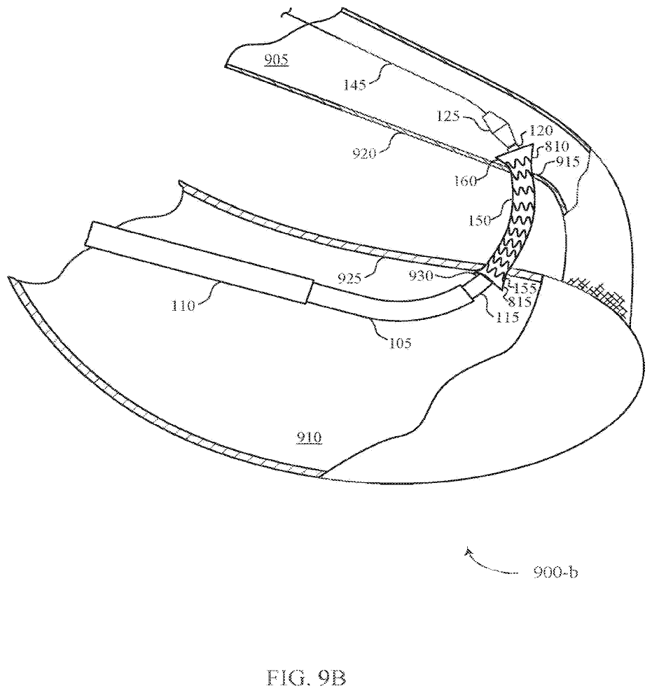

FIG. 1 illustrates a system 100 for providing access to a body lumen and delivering a stent in accordance with aspects of the present disclosure. The system 100 generally includes an outer sheath 105, an isolation sheath 110, a proximal marker 115, an anchoring component 120, an electrocautery tip 125, an inner tubular member 130, a stent 150, a middle member 165, and a guidewire 145 (FIGS. 9A and 9B). The system 100 can be provided as individual components, selectively combined components, or all together as a kit of components.

During a luminal access and stent 150 delivery procedure, the electrocautery tip 125 may access the target body lumen by piercing a wall of the body lumen, for example, to deliver a stent 150. In general, a stent 150 is a frame or scaffolding structure sized for placement within a body lumen and configured to provide structural support to the inner surface of the body lumen. A stent 150 may be used to restore patency across narrowed or blocked areas within the body lumen due to inflammation, tumors, plaque buildup, or any other obstructive feature. Although references to the pancreaticobiliary system are provided herein, it should be appreciated that the stents described herein may be used in any body lumen. Furthermore, as discussed in more detail below, the stent 150 may be disposed around the inner tubular member 130.

The stent 150 may be made from any number of materials, combinations of materials, and constructions. In some examples, the stent 150 is a self-expanding stent. The stent 150 may be a wire-form stent formed by one or more helically wrapped wires. However, it should be appreciated that the stent 150 may be made from other stent constructions or combinations of stent constructions. In other examples, the stent 150 is a laser-cut stent formed from a single metallic tube with regions cut away for increased flexibility. In yet other examples, the stent 150 is a braided stent made from a plurality of wires joined together in a cross-hatch configuration. In some examples, the stent 150 may be a combination of the braided stent and the wire-form stent.

It may be appreciated that the different stent constructions may exhibit particular characteristics such as radial expansive force, flexibility, reduced foreshortening, or migration resistance that may render a certain construction advantageous for a particular use. For example, the helical wrapping pattern of the stent 150 may be configured to reduce a foreshortening of the stent body upon deployment from an undeployed configuration to the deployed configuration to less than ten percent of a length of the stent body in the undeployed configuration. In such cases, the stent 150 may be an example of a non-foreshortening stent.

The individual wires or frame of the stent 150 may be made from any number of metallic materials including, but not limited to, titanium, nitinol, or stainless steel. It should be appreciated that other metallic or non-metallic materials may be used to construct the stent 150 that provides suitable flexibility, stiffness, and biocompatibility. The stent 150 may include a polymeric or fabric sleeve (e.g., first material) that covers some or all of the surface of the stent 150. Such a sleeve may protect the inner surface of the body lumen from the bare metal of the stent 150 and may prevent tissue ingrowth. For example, the stent 150 may include a helical wrapping pattern that is at least partially covered with a first material. In some examples, the stent 150 is a drug-eluting stent.

The outer sheath 105 of the system 100 has an elongate tubular body and an internal lumen extending from its proximal end to the distal end. In general, the outer sheath 105 may be configured to access a body lumen and to provide a conduit through which one or more devices (e.g., a guidewire 145) may pass to facilitate subsequent treatment of the body lumen or associate organs. The outer sheath 105 may include features that facilitate the direction-controlled delivery of a guidewire 145 within the body lumen for subsequent delivery of a stent 150, a biopsy device, a medicinal delivery element, or any number of other treatment or diagnostic devices.

The outer sheath 105 may be disposed coaxially along at least a portion of the inner tubular member 130 such that the stent 150 is disposed between the inner tubular member 130 and the outer sheath 105 while the stent 150 is in an undeployed configuration. The undeployed configuration may be an example of a stent 150 constrained within the outer sheath 105, an unexpanded configuration of the stent 150, or both. In some cases, the outer sheath 105 may include a braided extrusion in which a braided, distal section of the outer sheath 105 is fused with a braided, proximal section of the outer sheath 105. In some examples, the outer sheath 105 may include a coiled extrusion in which a coiled, distal section of the outer sheath 105 is fused with a coiled, proximal section of the outer sheath 105. In some cases, the outer sheath 105 may include a constant diameter along the length of the outer sheath 105.

In some examples, the outer sheath 105 may include a lubrication coating disposed within an inner surface of the outer sheath 105. The lubrication coating may be made from a variety of materials, including but not limited to silicone. In such cases, the lubrication coating may reduce deployment forces by at least thirty percent to ensure accurate placement of the stent 150 within the body lumen. In some cases, the lubrication coating of the outer sheath 105 may reduce the friction between the outer sheath 105 and the stent 150 as the outer sheath 105 is retracted over the stent 150. The isolation sheath 110 may be configured to receive the outer sheath 105 as the outer sheath 105 is retracted. The isolation sheath 110 may include a lubrication coating disposed within an inner surface of the isolation sheath 110.

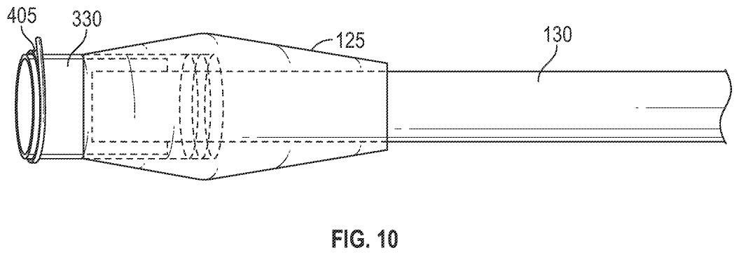

The inner tubular member 130 is generally an elongate, tubular member with a proximal end 135 and distal end 140 and is dimensioned to be advanced through the internal lumen of the outer sheath 105. The inner tubular member 130 may be configured to advance through an access site in a wall of the body lumen. In certain embodiments, the inner tubular member 130 includes one or more internal lumens extending from the proximal end 135 to the distal end 140 to house a power wire coupled with the electrocautery tip 125 in one internal lumen and the guidewire 145 in another internal lumen. The inner tubular member 130 may extend from a distal end or a proximal end of the electrocautery tip 125 to a lure at the handle end and provide a passageway for the guidewire 145. A distal end of the inner tubular member 130 may be disposed proximal to a distal edge of the electrocautery tip 125, be flush with the distal edge of the electrocautery tip 125, or extend distally beyond the distal edge of the electrocautery tip 125. As described below, the inner tubular member 130 is configured to house the guidewire 145. The inner tubular member 130 may be made of a number of thermally resistant materials including, but not limited to, polyether ether ketone (PEEK), polytetrafluoroethylene (PTFE), polyimide, or both. In aspects of the present disclosure, a distal end portion of the inner tubular member 130 is received within a thermally resistant tubular sleeve 330 (FIG. 10) having thermally resistant properties such that the inner tubular member 130 and the tubular sleeve 330 form a distal end portion of the inner tubular member 130 to prevent thermal damage to the inner tubular member 130. In aspects of the present disclosure, the tubular sleeve 330 may be formed of PTFE. In other aspects of the present disclosure, the tubular sleeve 330 may be formed of silicone. Still in other aspects of the present disclosure, the tubular sleeve 330 may be formed of ceramic or a ceramic impregnated material.

The inner tubular member 130 may be coupled with an anchoring component 120 at the distal end 140 of the inner tubular member 130. In certain embodiments, the distal end 140 of the inner tubular member 130 includes a tip or bulged portion (e.g., anchoring component 120). The stent 150 may be coupled to the inner tubular member 130 and the anchoring component 120. For example, the stent 150 may be concentric with the inner tubular member 130 and the anchoring component 120. As such, the inner tubular member 130 may extend through the lumen of the stent 150. For example, the stent 150 may be disposed coaxially onto the inner tubular member 130. The stent 150 may be positioned between the outer sheath 105 and the inner tubular member 130 at the proximal end 135 of the inner tubular member 130.

The middle member 165 is disposed coaxially onto the inner tubular member 130 such that the middle member 165 is positioned between the outer sheath 105 and the inner tubular member 130. The middle member 165 is disposed proximal to the proximal marker 115 such that a proximal end of the proximal marker 115 abuts a distal end of the middle member 165. In aspects of the present disclosure, the distal end of the middle member 165 may be coupled to the proximal end of the proximal marker 115 via suitable coupling features (e.g., disposed on the proximal marker 115 and/or the middle member 165) for improved transitioning from one component to the other.

The anchoring component 120 may extend through the lumen of the stent 150 at a distal portion 160 of the stent 150. In such cases, the stent 150 may be positioned between the outer sheath 105 and the anchoring component 120 at the distal end 140 of the inner tubular member 130. For example, the distal portion 160 of the stent 150 may be disposed coaxially along the anchoring component 120 such that the distal portion 160 of the stent 150 is disposed between the anchoring component 120 and the outer sheath 105 while the stent 150 is in the undeployed configuration. In aspects of the present disclosure, the anchoring component 120 may be formed by a proximal end portion of the electrocautery tip 125.

The anchoring component 120 may be made from a variety of materials, including but not limited to silicone. The anchoring component 120 may be disposed at a distal end 140 of the inner tubular member 130 and configured to retain a distal portion 160 of the stent 150 in place along the inner tubular member 130 as the outer sheath 105 is retracted proximally to deploy the stent 150. As described below in further detail, upon retraction of the outer sheath 105, the stent 150 may release from the anchoring component 120 and expand into a deployed configuration within the body lumen. The deployed configuration may be an example of an unconstrained configuration, an expanded configuration, or both.

The anchoring component 120 may be an example of a bump, an increased diameter component of the inner tubular member 130, a hook, or a combination thereof. In such cases, the anchoring component 120 may be configured to keep the distal portion 160 of the stent 150 stationary as the outer sheath 105 is retracted. In some examples, the anchoring component 120 may be 8 mm in length or 4 cm in length for a stent 150 that is 8-10 cm in length. In some cases, the inner tubular member 130 may include a single anchoring component 120 or more than one anchoring component 120. For example, the inner tubular member 130 may include at least three anchoring components 120 made of polyether block amide (PEBA) and positioned along the inner tubular member 130. The anchoring components 120 may extend 1 cm higher from the outer surface of the inner tubular member 130.

The system 100 may further include a proximal marker 115. The proximal marker 115 may be an example of a proximal marker that is disposed around the inner tubular member 130 and positioned such that a proximal end of the stent 150 abuts against the proximal marker 115 while the stent 150 is in the undeployed configuration. The proximal marker 115 may be configured to indicate a location of the proximal end of the stent 150 within the system 100. The proximal marker 115 may be configured to retain a proximal portion 155 of the stent 150 in place along the inner tubular member 130 as the outer sheath 105 is retracted proximally to deploy the stent 150. In some cases, the proximal marker 115 may be coupled with the middle member 165 and the inner tubular member 130 such that the proximal marker 115 remains stationary as the outer sheath 105 is retracted.

The proximal marker 115 includes generally an elongate, tubular member and is configured to house the inner tubular member 130. In some cases, a distal end portion of the proximal marker 115 may be tapered or stepped such that a distal end of the proximal marker 115 may extend underneath the proximal portion 155 of the stent 150. For example, the proximal marker 115 may be an example of a proximal anchoring component such that the proximal marker 115 may be configured to compress the proximal portion 155 of the stent 150 between the proximal marker 115 and the outer sheath 105.

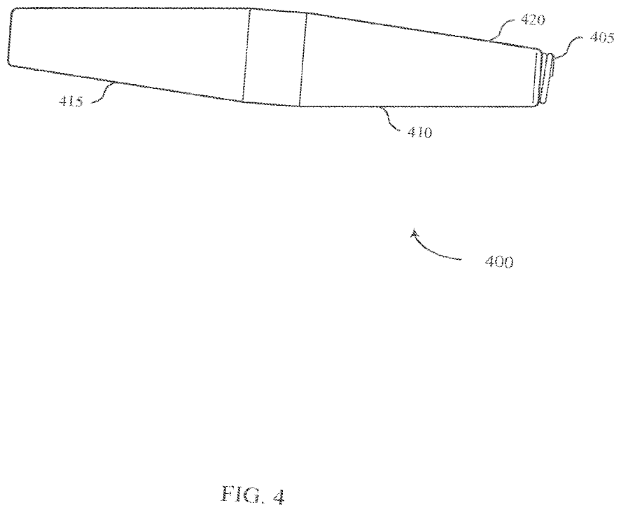

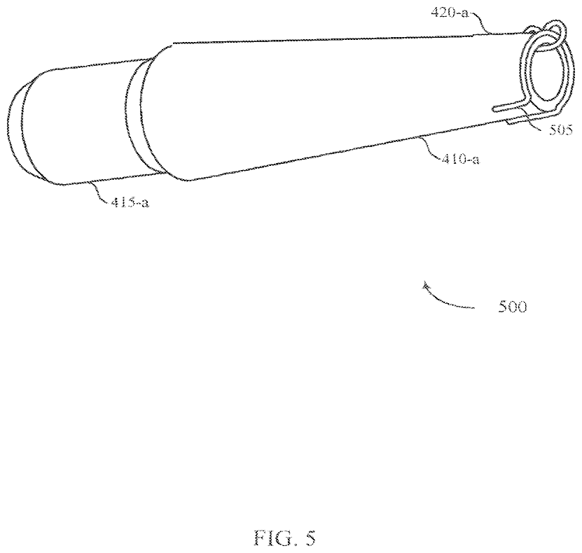

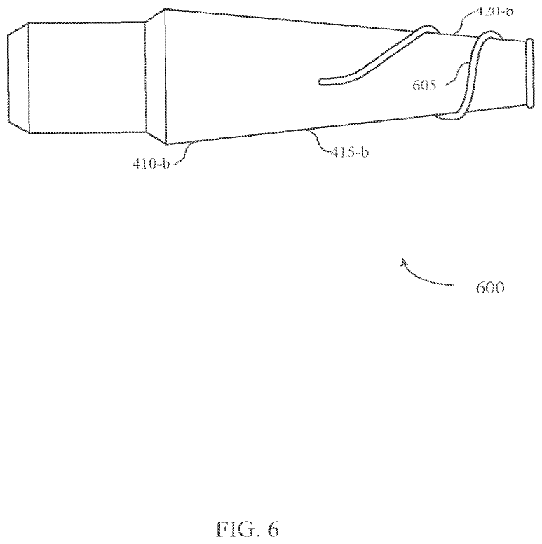

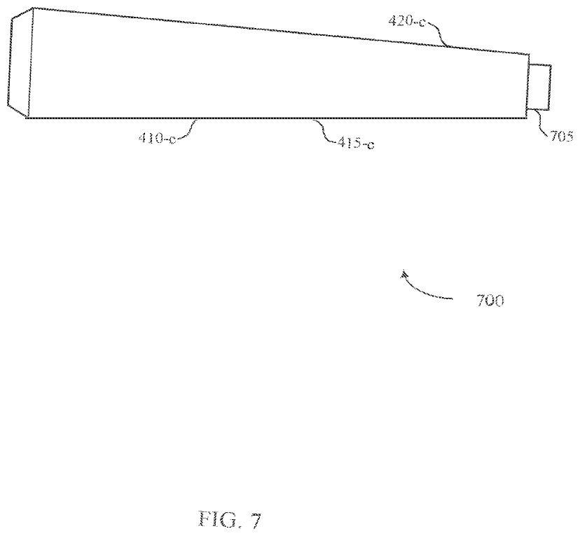

The electrocautery tip 125 may be an example of a distal cutting element coupled with a distal end portion of the inner tubular member 130 and configured to create the access site in the wall of the body lumen. The electrocautery tip 125 includes a coiled electrode wire that extends radially around a circumference of a distal end of the electrocautery tip 125 (FIG. 4), a single electrode wires that extends longitudinally and in a proximal direction from a distal end of the electrocautery tip 125 (FIG. 5), a single, spiral electrode wire that extends around a distal end of the electrocautery tip 125 (FIG. 6), or an electrode tube (FIG. 7). In some cases, the electrocautery tip 125 may include a tapered cover disposed around the electrocautery tip 125. The outer sheath 105 may at least partially overlap the tapered cover. The tapered cover may be made from a variety of materials, including but not limited to ceramic or a flexible material such as silicone. In some cases, the outer diameter of the electrocautery tip 125 may be equal to the inner diameter of the outer sheath 105. In other examples, the outer diameter of the electrocautery tip 125 may be greater than the inner diameter of the outer sheath 105.

The guidewire 145 is generally a flexible elongate member configured to slidably advance through the internal lumen of the inner tubular member 130. In such cases, the guidewire 145 may be disposed within the inner tubular member 130. The guidewire 145 may be uniform in size and stiffness along its entire length, or alternatively, may include sections of differing stiffness. The guidewire 145 may be insulated with one or more materials having dielectric strength and lubricity. Such materials may be laminated, coated, or heat shrunk over a core of the guide wire 145.

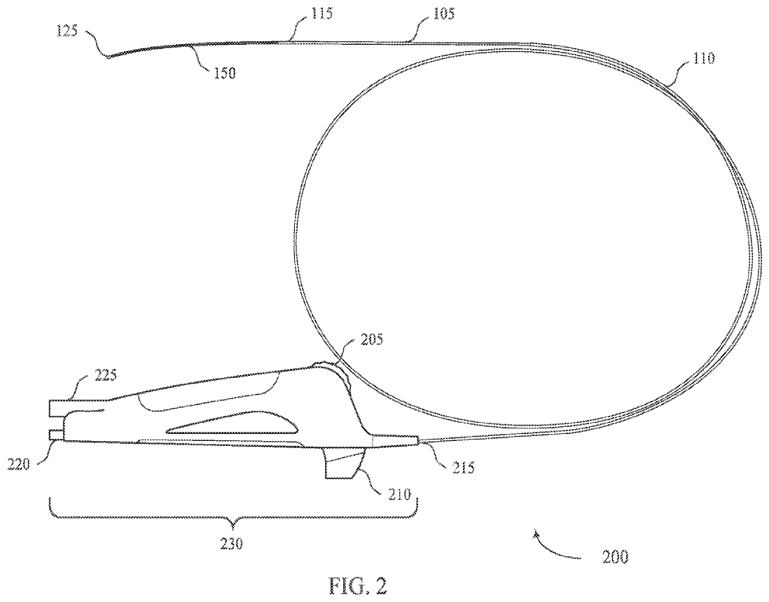

FIG. 2 illustrates a system 200 for providing access to a body lumen in accordance with aspects of the present disclosure. The system 200 generally includes the outer sheath 105, the isolation sheath 110, the proximal marker 115, the anchoring component 120 (not shown), the electrocautery tip 125, the inner tubular member 130 (not shown), and the stent 150, which may be examples of the corresponding components described with reference to FIG. 1. The system 200 may also include a thumbwheel 205, a stationary member 210, a guidewire lumen luer 220, and a connector port 225. The system 200 can be provided as individual components, selectively combined components, or all together as a kit of components.

The thumbwheel 205 may be coupled with a proximal end 215 of the outer sheath 105 and configured to retract the outer sheath 105 proximally. In some cases, the outer sheath 105 may be bonded (e.g., attached) to the thumbwheel 205 via a cable and a carriage (e.g., a plastic component). For example, the outer sheath 105 may be coupled with the handle assembly 230 via the carriage at the proximal end 215 of the outer sheath 105. The actuation of the thumbwheel 205 may retract the outer sheath 105 to deploy the stent 150. As described below, the outer sheath 105 may be retracted into the stationary isolation sheath 110.

The stationary member 210 may be an example of a locking pin. The stationary member 210 may be configured to prevent deployment when the device is inserted into the scope. For example, features of the stationary member 210 may engage into the carriage to prevent accidental deployment. To begin actuation of the thumbwheel 205, the stationary member 210 may be removed from the handle assembly 230. In such cases, the device may be deployed based on removing the stationary member 210 and actuating the thumbwheel 205.

In some cases, the isolation sheath 110 may stabilize the device against the scope channel and isolate friction that the catheter may experience otherwise in tortuosity. In such cases, the system 200 may enable accurate deployment of the stent 150 through the system 200 (e.g., including at least the isolation sheath 110 and the outer sheath 105) to ensure stability of the catheter against the scope. In some cases, the isolation sheath 110 may include two different diameters along the length of the isolation sheath 110 to customize interaction with the scope channel.

The isolation sheath 110 may be made of a material such as high-density polyethylene (HDPE). The isolation sheath 110 may be in contact with the inner diameter of the working channel of the endoscope, thereby inducing friction between the isolation sheath 110 and working channel. As the thumbwheel is actuated the outer sheath 105 is retracted, the friction between the isolation sheath 110 and the working channel may be greater than the friction between the isolation sheath 110 and the outer sheath 105 such that the isolation sheath 110 remains stationary as the outer sheath 105 is retracted. In such cases, the isolation sheath 110 may be configured as a barrier between the outer sheath 105 and the working channel, thereby reducing the overall friction on the system 200.

The outer sheath 105 may be inserted into a handle assembly 230, and once assembled, the outer sheath 105 extends through the handle assembly 230 to the target body lumen. In some cases, a power wire may be connected to the electrocautery tip 125 and may be laminated on the inner lumen member by a polyester heat shrink or covered by a thin tubing made from, for example, polyimide or other suitable materials. In such cases, the power wire may extend through the handle assembly 230 and to the connector port 225. The electrode of the electrocautery tip 125 may be connected to the connector port 225 in the handle assembly 230 through the power wire. In some cases, the electrode may connect to a radiofrequency (RF) generator through the connector port 225.

The guidewire lumen luer 220 may generally be a tubular structure that is sized to connect with the inner tubular member 130 to deploy the stent 150 within the body lumen. The guidewire lumen luer 220 may provide access to deliver the guide wire 145 and/or fluid injection to or from the human body through the working channel of an endoscope, for example. A proximal end of the middle member 165 (not shown) may be disposed within the guidewire lumen luer 220. As will be appreciated, the guidewire lumen luer 220 may be made from any number of biocompatible materials or combinations of materials suitable for medical sheaths, catheters, and the like.

In aspects of the present disclosure, a suitable contrast delivery accessory (not shown) such as, for example, a Y Connector (Hemostasis Valve Y Connector/Tuohy Borst Y-connector), may be included with the system 200 for coupling to the guidewire lumen luer 220 to facilitate contrast injection using suitably-sized syringes through the internal lumen of the inner tubular member 130 while the guidewire 145 is in place within a body lumen. In this scenario, the injected contrast passes through the internal lumen of the inner tubular member 130 and is expelled from the distal end of the electrocautery tip 125 into the body lumen.

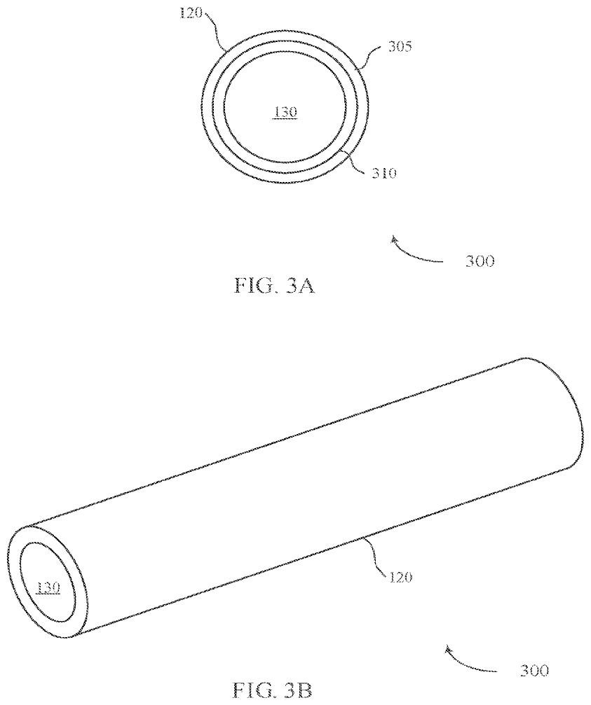

FIG. 3A illustrates a cross-sectional view of an anchoring component 300 in accordance with aspects of the present disclosure. The anchoring component 300 may be made from two different portions, each made from different materials, such as a first tubular member 305 and a second tubular member 310. The anchoring component 300 may be designed to retain a distal portion of a stent (not shown) in place along the inner tubular member 130 as the outer sheath (not shown) is retracted proximally to deploy the stent. In aspects of the present disclosure, the anchoring component 300 may be formed by a proximal end portion of the electrocautery tip 125 (FIGS. 1 and 2).

The anchoring component 300 may be disposed onto a distal portion of the inner tubular member 130 such that the anchoring component 300 is disposed coaxially along the inner tubular member 130. In such cases, the anchoring component 300 is disposed between the inner tubular member 130 and the distal portion of the stent. The anchoring component 300 may be an example of a anchoring component 120 described with reference to FIGS. 1-2. In accordance with various examples, anchoring component 300 may be used to retain a distal portion of the stent in place along the inner tubular member as the outer sheath is retracted proximally to deploy the stent, as described with reference to FIGS. 9A and 9B.

In some cases, the first tubular member 305 may be disposed within the tubular body of the anchoring component 300, and the second tubular member 310 may be disposed outside the tubular body of the anchoring component 300. In such cases, the first tubular member 305 may line the full inner circumference of the tubular body of the anchoring component 300. The first tubular member 305 may contact the inner tubular member 130. The second tubular member 310 may line the full outer circumference of the tubular body of the anchoring component 300. The second tubular member 310 may contact the distal portion of the stent. The length of the first tubular member 305 and the second tubular member 310 may each be equal to the length of the anchoring component 300.

The first tubular member 305 may include a first material, and the second tubular member 310 may include a second material. Exemplary materials of the first material and the second material include, but are not limited to, PEBA. The first material of the first tubular member 305 may include a first durometer value, and the second material of the second tubular member 310 may include a second durometer value different than the first durometer value. For example, the first durometer value may be higher than the second durometer value. In other examples, the first durometer value and the second durometer value may be the same.

The first material of the first tubular member 305 may include a higher durometer value as compared to the durometer value of the second material of the second tubular member 310 such that the first tubular member 305 resists compression as the outer sheath is retracted. The second material of the second tubular member 310 may include a lower durometer value as compared to the durometer value of the first material of the first tubular member 305 such that the second tubular member 310 increases the tackiness and grip on the distal portion of the stent. In such cases, as the outer sheath is retracted, the distal portion of the stent remains in place along the inner tubular member 130 as the outer sheath is retracted proximally. In some cases, as the outer sheath is retracted over the distal portion of the stent, the second tubular member 310 may compress axially while the first tubular member 305 may refrain from compressing axially. In such cases, the first tubular member 305 may maintain axial stiffness as the outer sheath is retracted.

FIG. 3B illustrates a perspective view of an anchoring component 300 in accordance with aspects of the present disclosure. The anchoring component 300 may be manufactured by coextruding the first material of the first tubular member 305 and the second material of the second tubular member 310 to a tubular body of the anchoring component 300. In some cases, the anchoring component 300 including the first tubular member 305 and the second tubular member 310 may be fused together using hot air or a glow ring. To bond the first tubular member 305 to the second tubular member 310, the tubular body of the anchoring component 300 may be reflowed. In some examples, the inner diameter of the anchoring component 300 may be 0.059 inches, and the outer diameter of the anchoring component 300 may be 0.066 inches.

The inner tubular member 130 may be bonded to the anchoring component 300. In some cases, the inner tubular member 130 may be bonded to the first tubular member 305. For example, the anchoring component 300 may be loaded onto the inner tubular member 130, and the anchoring component 300 and the inner tubular member 130 may be fused together (e.g., cured together) using hot air or a glow ring.

As the outer sheath is retracted, the anchoring component 300 may remain in a locked position (e.g., stationary). For example, the inner tubular member 130 may remain in the locked position as the outer sheath is retracted. In such cases, the anchoring component 300 may maintain a cylindrical form and in place along the inner tubular member 130 based on the dual-durometer values of the materials of the anchoring component 300 and the bond between the inner tubular member 130 and the anchoring component 300.

In some cases, the anchoring component 300 may be an example of an increased diameter portion of the inner tubular member 130. The anchoring component 300 may be an example of a hook, a bump, or other feature disposed at a distal portion of the inner tubular member 130 and configured to retain a distal portion of the stent in place along the inner tubular member 130 as the outer sheath is retracted proximally to deploy the stent.

FIG. 4 illustrates a distal cutting element 400 with a coiled electrode wire 405 in accordance with aspects of the present disclosure. The distal cutting element 400 may include a coiled electrode wire 405, an electrocautery tip 410, and a cover 415. A distal tip of the inner tubular member 130 (FIG. 1) may be disposed proximal to, be flush with, or be disposed distal to a distal edge of the coiled electrode wire 405. The electrocautery tip 410 may be configured to apply energy to a wall of a body lumen. Based on applying the energy, the body lumen may be accessed via the access site to position a stent within the body lumen.

The electrocautery tip 410 may include a tapered cover 415 disposed around the electrocautery tip 410. In some cases, the outer sheath (not shown) may at least partially overlap the tapered cover 415. The cover 415 may be configured to house the coiled electrode wire 405. The cover 415 may be made of a number of materials including, but not limited to silicone, a ceramic material, PTFE, a dielectric material, or a combination thereof. In some cases, the cover 415 may include barium sulfate to improve fluoroscopy and echo visibility. Based on the materials of the cover 415, the materials may be adhesive bonded, inserted molded, heat bonded, or a combination thereof. In some cases, the cover 415 may be made of a flexible material such that the electrocautery tip 410 may be maneuvered through the body lumen to the access site.

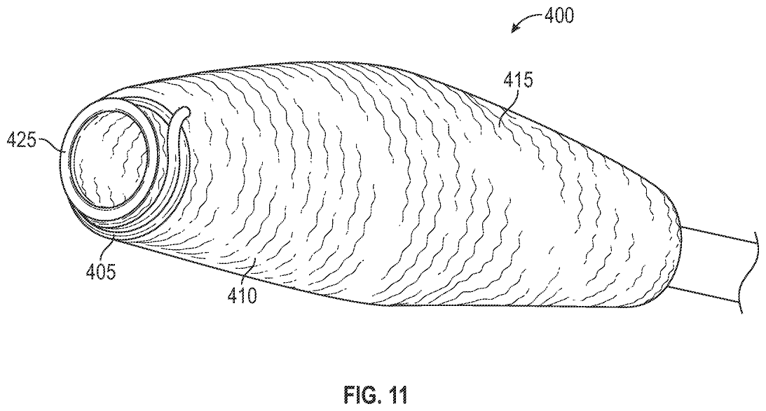

In some cases, the outer diameter of the cover 415 may be 0.072 inches. The cover 415 may include a single taper. For example, the single taper may be located at a distal end of the cover 415. In some cases, the cover 415 may include a double taper that tapers from a midpoint of the cover 415 and to a proximal end of the cover 415 and from the midpoint of the cover 415 and to the distal end of the cover 415. In aspects of the present disclosure, the distal edge of the cover 415 may include a distal extension 425 (FIG. 11) that is stepped down relative to the rest of the cover 415. As shown in FIG. 11, the coiled electrode wire 405 may be looped around the distal extension 425. A distal end of the extension may be flush with a distal edge of the coiled electrode wire 405 or may extend distally of the distal edge of the coiled electrode wire 405.

In some cases, the electrocautery tip 410 includes the coiled electrode wire 405. The coiled electrode wire 405 may extend radially around a circumference of a distal end 420 of the electrocautery tip 410. The coiled electrode wire 405 may be made of a number of metallic materials, but not limited to copper. In some cases, the coiled electrode wire 405 may include a single coil loop or may include more than one coil loop (e.g., a double coil loop). In aspects of the present disclosure, the coiled electrode wire 405 may include a single coil loop or a plurality of coil loops, which extend radially around a circumference of the distal end 420. The distal tip of the inner tubular member 130 may be disposed proximal to, flush with, or extending through the coiled electrode wire 405 such that the distal tip of the inner tubular member 130 is disposed distal to the coiled electrode wire 405. The coiled electrode wire 405 may extend through a lumen of the electrocautery tip 410 and extend from the distal end 420 of the electrocautery tip 410 to the proximal end of the electrocautery tip 410. The coiled electrode wire 405 may be exposed at the distal end 420 of the electrocautery tip 410 such that the coiled electrode wire 405 may be used as a distal cutting element to pierce the body lumen and cut the tissue of the body lumen. The coiled electrode wire 405 may be monopolar or bipolar.

The coiled electrode wire 405 may include a return wire that extends longitudinally through a lumen of the cover 415. For example, the return wire may be a straight electrode wire that is concentric with the cover 415. As such, the coiled electrode wire 405 (e.g., return wire) may extend through the lumen of the cover 415. For example, the cover 415 may be disposed coaxially onto the coiled electrode wire 405. The inner tubular member (not shown) may be positioned between the coiled electrode wire 405 and the cover 415. In some cases, the return wire of the coiled electrode wire 405 may be positioned between the inside surface of the cover 415 and the inner lumen member. In aspects of the present disclosure, the return wire of the coiled electrode wire 405 may be at least partially formed (e.g., a proximal end portion) into one or more coil loops (not shown) for electrical connection to the power wire and/or for attachment to a distal end portion of the inner tubular member 130.

In some cases, the inner lumen member may include a jacketed cable tube to serve as a coiled electrode and to insulate the inner lumen member and increase flexibility of the distal end 420 of the electrocautery tip 410. In aspects of the present disclosure, the inner lumen member may be disposed proximal to, flush with, or disposed distal to a distal edge of the jacketed cable tube. At least a portion of the jacketed cable tube (e.g., one or two coils) may be exposed (e.g., uninsulated) and may extend radially around a circumference of the distal end 420 of the electrocautery tip 410. In some cases, the jacketed cable tube may be fused, combined, co-extruded, or a combination thereof with the inner lumen member. The jacketed cable tube may be made of a number of materials, including but not limited to, a spring guide, a close-wound spring, a close coil, or a combination thereof using round or flat wire. The proximal end of the jacketed cable tube may be connected to connector port, as described with reference to FIG. 2, to allow for both fluid connection and electrical connection. In some examples, the inner lumen member may be bonded to the cover 415.

FIG. 5 illustrates a distal cutting element 500 with single electrode wires 505 in accordance with aspects of the present disclosure. The distal cutting element 500 may include single electrode wires 505, an electrocautery tip 410-a, and a cover 415-a. A distal tip of the inner tubular member 130 (FIG. 1) may be disposed proximal to, be flush with, or be disposed distal to a distal edge of the electrode wires 505. The electrocautery tip 410-a may be configured to apply energy to a wall of a body lumen. Based on applying the energy, the body lumen may be accessed via the access site to position a stent within the body lumen.

The electrocautery tip 410-a may include a tapered cover 415-a disposed around the electrocautery tip 410-a. In some cases, the outer sheath (not shown) may at least partially overlap the tapered cover 415-a. The cover 415-a may be configured to house the single electrode wires 505. The cover 415-a may be made of a number of materials including, but not limited to silicone, a ceramic material, PTFE, a dielectric material, or a combination thereof. In some cases, the cover 415-a may include barium sulfate to improve fluoroscopy and echo visibility. Based on the materials of the cover 415-a, the materials may be adhesive bonded, inserted molded, heat bonded, or a combination thereof. In some cases, the cover 415-a may be made of a flexible material such that the electrocautery tip 410-a may be maneuvered through the body lumen to the access site.

In some cases, the cover 415-a may include a single taper. For example, the single taper may be located at a distal end of the cover 415-a. In some cases, the cover 415-a may include a stepped double taper. The stepped double taper may taper from a proximal end of the electrocautery tip 410-a to a stepped level and then from the stepped level to the distal end 420-a of the electrocautery tip 410-a.

In some cases, the electrocautery tip 410-a includes the single electrode wires 505. The single electrode wire 505 may extend radially around a circumference of a distal end 420-a of the electrocautery tip 410-a and then extend longitudinally and in a proximal direction from the distal end 420-a of the electrocautery tip 410-a. For example, the two electrodes ends of the single electrode wire 505 may be straight and extend longitudinally and in a proximal direction from the distal end 420-a of the electrocautery tip 410-a. In some cases, the two electrode ends may be an example of a “s” shaped curve. In some examples, the single electrode wire 505 may be an example of a dome-shaped electrode, a flat wire electrode, or a stamped sheet electrode. The single electrode wires 505 may be monopolar or bipolar.

The single electrode wires 505 may be made of a number of metallic materials, but not limited to copper. In some cases, the single electrode wire 505 may extend through a lumen of the electrocautery tip 410-a and extend from the distal end 420-a of the electrocautery tip 410-a to the proximal end of the electrocautery tip 410-a. The single electrode wire 505 may be exposed at the distal end 420-a of the electrocautery tip 410-a such that the single electrode wires 505 may be used as a distal cutting element to pierce the body lumen and cut the tissue of the body lumen.

The single electrode wires 505 may include a return wire that extends longitudinally through a lumen of the cover 415-a. For example, the return wire may be a straight electrode wire that is concentric with the cover 415-a. As such, the single electrode wire 505 (e.g., return wire) may extend through the lumen of the cover 415-a. For example, the cover 415-a may be disposed coaxially onto the coiled electrode wire 405-a. The inner tubular member (not shown) may be positioned between the single electrode wire 505 and the cover 415-a. In some cases, the return wire of the single electrode wire 505 may be positioned between the inside surface of the cover 415-a and the inner lumen member. In some cases, the inner lumen member may include a jacketed cable tube to insulate the inner lumen member and increase flexibility of the distal end 420-a of the electrocautery tip 410-a.

FIG. 6 illustrates a distal cutting element 600 with a spiral electrode wire 605 in accordance with aspects of the present disclosure. The distal cutting element 600 may include a spiral electrode wire 605, an electrocautery tip 410-b, and a cover 415-b. A distal tip of the inner tubular member 130 (FIG. 1) may be disposed proximal to, be flush with, or be disposed distal to a distal edge of the coiled electrode wire 605. The electrocautery tip 410-b may be configured to apply energy to a wall of a body lumen. Based on applying the energy, the body lumen may be accessed via the access site to position a stent within the body lumen.

The electrocautery tip 410-b may include a tapered cover 415-b disposed around the electrocautery tip 410-b. In some cases, the outer sheath (not shown) may at least partially overlap the tapered cover 415-b. The cover 415-b may be configured to house the spiral electrode wire 605. The cover 415-b may be made of a number of materials including, but not limited to silicone, a ceramic material, PTFE, a dielectric material, or a combination thereof. In some cases, the cover 415-b may include barium sulfate to improve fluoroscopy and echo visibility. Based on the materials of the cover 415-b, the materials may be adhesive bonded, inserted molded, heat bonded, or a combination thereof. In some cases, the cover 415-b may be made of a flexible material such that the electrocautery tip 410-b may be maneuvered through the body lumen to the access site. In some cases, the cover 415-b may include a single taper. For example, the single taper may be located at a distal end of the cover 415-b. The single taper may taper from a proximal end of the electrocautery tip 410-b to the distal end 420-b of the electrocautery tip 410-b.

In some cases, the electrocautery tip 410-b includes the spiral electrode wire 605. The spiral electrode wire 605 may extend radially around a distal end 420-b of the electrocautery tip 410-b. The spiral electrode wire 605 may be a single electrode wire. In some cases, the spiral electrode wire 605 may be combined with the coiled electrode wire. For example, the spiral electrode wire 605 may include at least two spirals around the distal end 420-b of the electrocautery tip 410-b and one or more coils of the coiled electrode wire at the distal end 420-b of the electrocautery tip 410-b. The spiral electrode wire 605 may run longitudinally down the cover 415-b. The spiral electrode wire 605 may be monopolar or bipolar.

The spiral electrode wire 605 may be made of a number of metallic materials, but not limited to copper. In some cases, the spiral electrode wire 605 may extend through a lumen of the electrocautery tip 410-b and extend from the distal end 420-b of the electrocautery tip 410-b to the proximal end of the electrocautery tip 410-b. The spiral electrode wire 605 may be exposed at the distal end 420-b and along a distal portion of the electrocautery tip 410-b such that the spiral electrode wire 605 may be used as a distal cutting element to pierce the body lumen and cut the tissue of the body lumen.

The spiral electrode wire 605 may include a return wire that extends longitudinally through a lumen of the cover 415-b. For example, the return wire may be a straight electrode wire that is concentric with the cover 415-b. As such, the spiral electrode wire 605 (e.g., return wire) may extend through the lumen of the cover 415-b. For example, the cover 415-b may be disposed coaxially onto the spiral electrode wire 605. The inner tubular member (not shown) may be positioned between the spiral electrode wire 605 and the cover 415-b. In some cases, the return wire of the spiral electrode wire 605 may be positioned between the inside surface of the cover 415-b and the inner lumen member. In some cases, the inner lumen member may include a jacketed cable tube to insulate the inner lumen member and increase flexibility of the distal end 420-b of the electrocautery tip 410-b.

FIG. 7 illustrates a distal cutting element 700 with an electrode tube 705 in accordance with aspects of the present disclosure. The distal cutting element 700 may include an electrode tube 705, an electrocautery tip 410-c, and a cover 415-c. A distal tip of the inner tubular member 130 (FIG. 1) may be disposed proximal to, be flush with, or be disposed distal to a distal edge of the coiled electrode tube 705. The electrocautery tip 410-c may be configured to apply energy to a wall of a body lumen. Based on applying the energy, the body lumen may be accessed via the access site to position a stent within the body lumen.

The electrocautery tip 410-c may include a tapered cover 415-c disposed around the electrocautery tip 410-c. In some cases, the outer sheath (not shown) may at least partially overlap the tapered cover 415-c. The cover 415-c may be configured to house the electrode tube 705. The cover 415-c may be made of a number of materials including, but not limited to silicone, a ceramic material, PTFE, a dielectric material, or a combination thereof. In some cases, the cover 415-c may include barium sulfate to improve fluoroscopy and echo visibility. Based on the materials of the cover 415-c, the materials may be adhesive bonded, inserted molded, heat bonded, or a combination thereof. In some cases, the cover 415-c may be made of a flexible material such that the electrocautery tip 410-c may be maneuvered through the body lumen to the access site. In some cases, the cover 415-c may include a single taper. For example, the single taper may be located at a distal end of the cover 415-c. The single taper may taper from a proximal end of the electrocautery tip 410-c to the distal end 420-c of the electrocautery tip 410-c.

In some cases, the electrocautery tip 410-c includes the electrode tube 705. The electrode tube 705 may extend radially around a circumference of the distal end 420-c of the electrocautery tip 410-c. The electrode tube 705 may be a single electrode tube coil. The electrode tube 705 may be monopolar or bipolar.

The electrode tube 705 may be made of a number of metallic materials, but not limited to copper, stainless steel, or both. In some cases, the electrode tube 705 may extend through a lumen of the electrocautery tip 410-c and extend from the distal end 420-c of the electrocautery tip 410-c to the proximal end of the electrocautery tip 410-c. The electrode tube 705 may be exposed at the distal end 420-c such that the electrode tube 705 may be used as a distal cutting element to pierce the body lumen and cut the tissue of the body lumen. In some cases, the electrode tube 705 may include 1 mm exposed at the distal end 420-c of the electrocautery tip 410-c.

The electrode tube 705 may extend through the lumen of the cover 415-c to act as a return wire that extends longitudinally through the lumen of the cover 415-c. For example, the electrode tube 705 may be concentric with the cover 415-c. As such, the electrode tube 705 may extend through the lumen of the cover 415-c. For example, the cover 415-c may be disposed coaxially onto the electrode tube 705. The inner tubular member (not shown) may be positioned between the electrode tube 705 and the cover 415-c. In some cases, the electrode tube 705 may be positioned between the inside surface of the cover 415-c and the inner lumen member. In some cases, the inner lumen member may include a jacketed cable tube to insulate the inner lumen member and increase flexibility of the distal end 420-c of the electrocautery tip 410-c.

In some cases, the electrocautery tip 410-c may include a metal dilator. The electrocautery tip 410-c may be an example of a dilator that may be energized during a one-step access operation after the guidewire is placed within the body lumen. The metal dilator may be a single element or may have two elements where the distal end of one or both of the elements serves as a cutting element. In some cases, the distal end of the metal dilator may be raised to improve cutting efficiency.

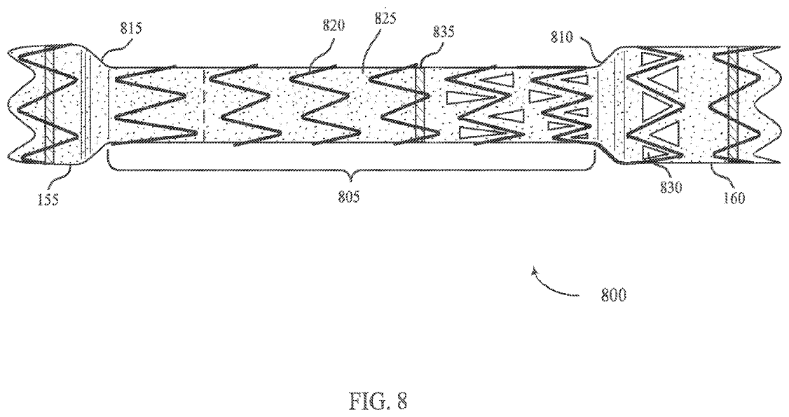

FIG. 8 illustrates a stent 800 in accordance with aspects of the present disclosure. The stent 800 may be configured to restore luminal flow across narrowed areas or blockages within a body lumen, as described with reference to FIG. 1. The stent 800 may be sized or otherwise adapted to be placed within any body lumen, such as those associated with the pancreaticobiliary system, the arterial system, the bronchial system, the urinary system, or any other luminal system that may require stent treatment. In some cases, the stent 800 may be placed within the body lumen by a stent delivery system, as described with reference to FIGS. 1 and 2. The stent 800 may be an example of stent 150 as described with reference to FIGS. 1 and 2.

The stent 800 may be categorized as having a proximal portion 155, which may, for example, be placed within a duodenum, and a distal portion 160 which may, for example, be placed within a biliary duct. The stent 800 may include a stent body 805 that has a diameter and a length in a deployed configuration. The stent body 805 may extend between the distal portion 160 and the proximal portion 155. The stent body 805 may be an example of a mid-body portion of the stent 800 that includes a narrow region between a first flared portion 810 and a second flared portion 815.

The stent 800 may include a first anchoring member (e.g., first flared portion 810) coupled with the distal portion 160 of the stent body 805. The first flared portion 810 may be configured to increase a diameter of the distal portion 160 of the stent body 805 to a second diameter greater than the first diameter. In such cases, the diameter of the first flared portion 810 may be greater than a diameter of the stent body 805 in the deployed configuration. The first flared portion 810 may be coupled with a distal end of the stent body 805 and spaced around a circumference of the distal end of the stent body 805.

The stent 800 may include a second anchoring member (e.g., a second flared portion 815 coupled with a proximal portion 155 of the stent body 805 and configured to increase a diameter of the proximal portion 155 of the stent body 805 to a second diameter greater than the first diameter. In such cases, the diameter of the second flared portion 815 may be greater than a diameter of the stent body 805 in the deployed configuration. The second flared portion 815 may be coupled with a proximal end of the stent body 805 and sp aced around a circumference of the proximal end of the stent body 805. The first flared portion 810 and the second flared portion 815 may include a helical wrapping pattern 820.

The first flared portion 810 and second flared portion 815 may respectively bridge each end of the stent 800 (e.g., the proximal end and the distal end) to the stent body 805. For example, the first flared portion 810 may bridge the stent body 805 with the proximal end of the stent 800. The second flared portion 815 may bridge the stent body 805 with the distal end of the stent 800. In some cases, the transition between the narrower diameter of the stent body 805 and the wider diameters of the first flared portion 810 and the second flared portion 815 may be gradual or steep. The first flared portion 810 and the second flared portion 815 may enable the stent 800 to resist migration within the body lumen by expanding from an undeployed configuration to a deployed configuration, as described with reference to FIGS. 9A and 9B. The stent body 805 may bridge the two body lumens, and the first flared portion 810 and the second flared portion 815 may act as an anti-migration tool to prevent the stent 800 from moving further into either body lumen. Using the first flared portion 810 and the second flared portion 815 as an anti-migration tool may be less invasive to the body tissue compared to other anti-migration techniques used in stents.

The stent 800 may include a helical wrapping pattern 820 that may be at least partially covered with a cover material 825. The helical wrapping pattern 820 may be configured to reduce a foreshortening of the stent body 805 upon deployment from the undeployed configuration to the deployed configuration to less than ten percent of a length of the stent body 805 in the undeployed configuration. In such cases, the length of the stent body 805 may be maintained before deployment and after deployment to ensure accurate and precise placement with the body lumen. The stent 800 may be an example of a non-foreshortening stent.

The helical wrapping pattern 820 may include a single wire. The single wire of the helical wrapping pattern 820 may be made from any number of metallic materials including, but not limited to, titanium, nitinol, or stainless steel. It should be appreciated that other metallic or non-metallic materials may be used to construct the stent 800 that provide suitable flexibility, stiffness, and biocompatibility. The single wire may be helically wrapped around the stent 800 such that the helical wrapping pattern 820 extends from the proximal portion 155 and to the distal portion 160. In some cases, using the single wire may improve the structural stability of the stent 800 as compared to a multi-wire stent. In some cases, the helical wrapping pattern 820 may enable the stent 800 to evenly withstand pressure across the entire body of the stent 800.

In some examples, the cover material 825 may fully cover the stent 800. For example, the cover material 825 may cover an entire portion of the stent body 805, the first flared portion 810, and the second flared portion 815. In some examples, the cover material 825 may at least partially cover the stent body 805, the first flared portion 810, the second flared portion 815, or a combination thereof. The cover material 825 may cover the helical wrapping pattern 820 to protect the body lumen from the metallic contact of the single wire of the helical wrapping pattern 820. In some examples, the cover material 825 may include cut-out drainage holes, such as the drainage holes 830. In some cases, the drainage holes 830 may enable fluid drainage into the body lumen, which may increase the efficiency of the stent 800. In some cases, the drainage holes 830 may enable drainage across a duct where stent placement may be desired. In some cases, the placement of the drainage holes 830 at the distal portion 160 of the stent 800 may enable bile drainage while also preventing food or other debris from travelling up the lumen of the stent 800, thereby causing an occlusion. The marker bands 835 may be placed at each section of the stent 800. For example, the marker bands 835 may be disposed around the stent body 840, the first flared portion 810, and the second flared portion 815. The marker bands 835 may aid in stent placement, as described with reference to FIG. 1.

The stent 800 may be made from any number of materials, combinations of materials, and constructions. In some examples, the stent 800 may be a laser-cut stent formed from a single metallic tube with regions cut way for increased flexibility. For example, the helical wrapping pattern 820 may include a laser cut frame. In some examples, the stent 800 may be a wire-formed stent formed by one or more helically wrapped wires. It may be appreciated that the different stent constructions may exhibit particular characteristics such as radial expansive force, flexibility, reduced foreshortening, or migration resistance that may render a certain construction advantageous for a particular use.

FIG. 9A illustrates a stent delivery system 900-a with a flared portion of the stent 150 deployed in accordance with aspects of the present disclosure. The stent delivery system 900-a may generally include the isolation sheath 110, the outer sheath 105, the anchoring component 120, the electrocautery tip 125, the guidewire 145, and the stent 150, which may be examples of the corresponding components described with reference to FIGS. 1 through 8.

The stent delivery system 900-a may be configured to place a stent 150 within a first body lumen 905 to restore luminal flow from a first body lumen 905 to a second body lumen 910, thereby bypassing narrowed areas or blockages within at least the first body lumen 905. The stent delivery system 900-a may be sized or otherwise adapted to place a stent within any body lumen, such as those associated with the pancreaticobiliary system, the arterial system, the bronchial system, the urinary system, or any other luminal system that may require stent treatment.

The illustrated portions of the system include the first body lumen 905 (e.g., a common bile duct), which drains bile from both the cystic duct (which drains from the gallbladder) and the common hepatic duct (which drains from the liver) into the second body lumen 910 (e.g., duodenum), where the bile mixes and reacts with digesting food. A clinician may advance an endoscope (e.g., an EUS endoscope) into the lumen of a patient's duodenum (e.g., second body lumen 910) to a position in which the bile ducts may be visualized (e.g., via endosonography). The clinician may then access the common bile duct (e.g., first body lumen 905) by advancing a separate access device (e.g., a FNA catheter) from a working channel of the endoscope, through the wall 925 of the duodenum (i.e., trans-duodenally), and then through the wall 920 of the common bile duct. The guidewire 145 may then be deployed through the access device and into the first body lumen 905. Once the guidewire 145 is in place, the access device may be withdrawn and the stent delivery system may be slid over the guidewire 145 for luminal access. In aspects of the present disclosure, the stent delivery system may cut through the luminal walls 925 and 920 by energizing the electrocautery tip 125 over an insulated guide wire (e.g., guide wire 145). In other aspects, the stent delivery system may cut through the luminal wall 925 and 920 by energizing electrocautery tip 125 directly without the insulated guide wire 145. In still other aspects, the stent delivery system may cut through luminal walls 925 and 920 by energizing the electrocautery tip 125, and the guide wire 145 may be advanced into the first body lumen 905. Once the guide wire 145 is advanced into the first body lumen 905, the stent delivery system may then be further advanced into the first body lumen 905 over the guidewire 145.

During a luminal access and stent delivery procedure, the electrocautery tip 125 may access the target lumen (e.g., first body lumen 905) by piercing a wall 920 of the first body lumen 905, for example. The electrocautery tip 125 may access the target lumen (e.g., first body lumen 905) by piercing a wall 925 of the second body lumen 910 prior to piercing the wall 920 of the first body lumen 905. In such cases, the electrocautery tip 125 may exit the second body lumen 910 and target access of the first body lumen 905. The first body lumen 905 may be an example of the biliary duct, and the second body lumen 910 may be an example of the duodenum.

In some cases, to access the first body lumen 905, the electrocautery tip 125 may apply energy to the wall 920 of the first body lumen 905 and access, via the access site 915, the first body lumen 905 based on applying the energy to the wall 920 of the first body lumen 905. In some cases, prior to accessing the first body lumen 905, the electrocautery tip 125 may apply energy to the wall 925 of the second body lumen 910 and access the first body lumen 905 based on applying the energy to the wall 925 of the second body lumen 910. For example, the electrocautery tip 125 may cut the tissue in contact with the electrode of the electrocautery tip 125. In aspects of the present disclosure, prior to accessing the first body lumen 905, the electrocautery tip 125 may apply energy to the wall 925 of the second body lumen 910 and to the wall 920 of the first body lumen, and access the first body lumen 905. In some aspects, prior to accessing the first body lumen 905, the electrocautery tip 125 may apply energy to the wall 925 of the second body lumen 910 and to the wall 920 of the first body lumen, and access the first body lumen 905. An insulated guide wire (e.g., guide wire 145) may be advanced into the first body lumen 905, and then the stent delivery system may be advanced into the first body lumen 905 over the insulated guidewire. In some cases, the insulated guidewire is placed in the first body lumen 905 through the walls 925 and 920 via another catheter (e.g., a FNA catheter). Prior to accessing the first body lumen 905, the electrocautery tip 125 may apply energy to the wall 925 of the second body lumen 910 and to the wall 920 of the first body lumen 905 over the insulated guidewire and access the first body lumen 905.

The stent delivery system 900-a may be configured for choledochoduodenostomy (CDS) and hepaticogastrostomy (HGS) procedures in which the stent 150 is implanted across two tissues layers (e.g., duodenum to common bile duct or stomach to intrahepatic duct). In some cases, the stent delivery system 900-a may be configured for transmittal biliary drainage. In such cases, the stent 150 may bridge between the second body lumen 910 (e.g., the duodenum) and a portion of the first body lumen 905 (e.g., the biliary duct) to create a bridge to bypass an obstruction. The obstruction may be an example of a distal malignant biliary obstruction that obstructs drainage. For example, the stent delivery system 900-a may be configured to provide access to at least the common biliary duct to facilitate subsequent procedures to treat narrowed areas or blockages within the bile duct and create a bypass around the narrowed areas or blockages within the bile duct to provide access to the stomach and from the biliary duct via the stent 150.

Prior to retracting the outer sheath 105, the anchoring component 120 may be positioned within the first body lumen 905, and the marker (not shown) may be positioned within the second body lumen 910 such that the stent 150 traverses the first body lumen 905 and the second body lumen 910. The outer sheath 105 may be disposed around the inner lumen member and the anchoring component 120. As such, the distal portion 160-b of the stent 150 is disposed between the anchoring component 120 and the outer sheath 105, and the proximal portion of the stent is disposed between the inner lumen member and the outer sheath 105.

Once the outer sheath 105 is retracted and removed through the access site 915, the distal portion 160 of the stent 150 may deploy. As the outer sheath 105 is withdrawn through the access site 915, the inner lumen member (not shown) and the anchoring component 120 may remain stationary, and the distal portion 160 of the stent 150 may be exposed within the first body lumen 905. Once the desired anatomical position of the stent 150 has been achieved within the first body lumen 905, the outer sheath 105 may be retracted.

The outer sheath 105 may be retracted proximally and past the anchoring component 120 disposed at a distal portion of an inner tubular member based on positioning the stent 150. For example, the distal portion 160 of the stent 150 may be disposed coaxially along the anchoring component 120 such that the distal portion 160 of the stent 150 is disposed between the anchoring component 120 and the outer sheath 105 while the stent 150 is in the undeployed configuration. The distal portion 160 of the stent 150 may be deployed from the outer sheath 105 into a deployed configuration within the first body lumen 905 based on retracting the outer sheath 105 past the anchoring component 120.

In some cases, the distal portion 160 of the stent 150 may be released from the anchoring component 120 and into the deployed configuration in response to retracting the outer sheath 105 past the anchoring component 120. For example, the distal portion 160 of the stent 150 may expand from the undeployed configuration to the deployed configuration while the outer sheath 105 is retracted based on a pressure being released between the anchoring component 120 and the outer sheath 105 as the outer sheath 105 is retracted. In such cases, the distal portion 160 of the stent 150 is no longer compressed between the outer sheath 105 and the anchoring component 120 and is free to expand within the first body lumen 905.

The distal portion 160 of the stent 150 may include the first flared portion 810. In such cases, the first flared portion 810 may expand within the first body lumen 905 in direct response to retracting the outer sheath 105 past the anchoring component 120. As the outer sheath 105 is removed through the access site 915, the distal portion 160 of the stent 150 expands to expose the first flared portion 810. As the distal portion 160 of the stent 150 expands, the first flared portion 810 contacts the wall 920 of the first body lumen 905. The first flared portion 810 (e.g., distal portion 160) of the stent 150 may be anchored within the first body lumen 905 such that the distal portion 160 of the stent 150 remains in a fixed position. In that case, the distal portion 160 prevents the stent 150 from being further withdrawn through the access site 915. The clinician may be able to feel the resistance of the first flared portion 810 against the first body lumen 905 and may therefore infer the location of the stent 150. Additionally or alternatively, the distal portion 160 of the stent 150 may be viewed under fluoroscopy or similar imaging techniques to infer the location of the stent 150.

The distal portion 160 of the stent 150 may be retained in place along the inner tubular member as the outer sheath 105 is retracted past the anchoring component 120 based on the distal portion 160 of the stent 150 being disposed (e.g., compressed) between the anchoring component 120 and the outer sheath 105. While the outer sheath 105 is retracted, the anchoring component 120 and the inner tubular member may be maintained in a locked position (e.g., stationary with respect to the withdrawn outer sheath 105). As the outer sheath 105 is retracted, the isolation sheath 110 is maintained in a locked position while retracting and withdrawing the outer sheath 105 into the isolation sheath 110.

The friction between the anchoring component 120 and the distal portion 160 of the stent 150 may keep the stent 150 in place along the inner tubular member as the outer sheath 105 is retracted. In some cases, the friction between the stent 150 and the outer sheath 105 may keep the stent 150 in place as the outer sheath 105 is retracted such that after the outer sheath 105 is retracted past the anchoring component 120 (e.g., clears the anchoring component 120), the distal portion 160 of the stent 150 expands from an undeployed to a deployed configuration.

In such cases, the outer sheath 105 may be retracted to a first position (e.g., past a distal end of the anchoring component 120), and the distal portion 160 of the stent 150 may deploy in a same location as compared to a location prior to retracting the outer sheath 105. Once the outer sheath 105 is retracted past the distal end of the anchoring component 120, the distal portion 160 of the stent 150 that was positioned between the outer sheath 105 and the anchoring component 120 may expand into the first body lumen 905 and anchor itself to the first body lumen 905. In such cases, the anchored distal portion 160 of the stent 150 may maintain the stent in a stationary position as the outer sheath 105 is retracted.

FIG. 9B illustrates a stent delivery system 900-b with the stent 150 fully deployed in accordance with aspects of the present disclosure. As the outer sheath 105 is further withdrawn proximally, the inner lumen member (not shown) may remain stationary, and the stent 150 may be exposed within the first body lumen 905 and into the second body lumen 910. To deploy the stent 150 within the first body lumen 905 and second body lumen 910, the outer sheath 105 may be retracted past a distal end 930 of the proximal marker 115. In the case of a non-foreshortening stent, the stent 150 expands to contact the inner surface of the first body lumen 905 and the inner surface of the second body lumen 910 such that the stent 150 forms a bridge between the first body lumen 905 and the second body lumen 910.

The distal end 930 the proximal marker 115 may be aligned with (typically some distance away from) the wall 925 of the second body lumen 910. While the outer sheath 105 is retracted, the proximal marker 115 may be maintained in a locked position after aligning (typically some distance away from) the distal end 930 of the proximal marker 115 with the wall 925 of the second body lumen 910. Once the outer sheath 105 is retracted past the distal end 930 of the proximal marker 115, the proximal portion 155 of the stent 150 may expand from within the outer sheath 105 in response to withdrawing the outer sheath 105 past the proximal marker 115. For example, the proximal portion 155 of the stent 150 may expand from within the outer sheath 105 such that upon fully exiting the outer sheath 105, the proximal portion 155 expands to a deployed configuration within the second body lumen 910. In such cases, the entire portion of the stent 150 may expand such that at least a portion of the stent 150 extends through the first body lumen 905 and into the second body lumen 910.

The proximal portion 155 of the stent 150 may be retained in place along the inner tubular member as the outer sheath 105 is retracted past the proximal marker 115 based on the proximal portion 155 of the stent 150 being disposed (e.g., compressed) between the inner lumen member and the outer sheath 105. In some cases, the proximal portion 155 of the stent 150 may be retained in place as the outer sheath 105 is retracted based on the proximal portion of the stent 150 abutting a proximal end of the proximal marker 115. While the outer sheath 105 is retracted, the inner tubular member may be maintained in a locked position (e.g., stationary with respect to the withdrawn outer sheath 105). As the outer sheath 105 is retracted, the isolation sheath 110 is maintained in a locked position while retracting and withdrawing the outer sheath 105 into the isolation sheath 110.

In such cases, the outer sheath 105 may be retracted to a second position (e.g., past the distal end 930 of the proximal marker 115), and the proximal portion 155 of the stent 150 may deploy in a same location as compared to a location prior to retracting the outer sheath 105. Once the outer sheath 105 is retracted past the distal end 930 of the proximal marker 115, the proximal portion 155 of the stent 150 that was positioned between the outer sheath 105 and the inner lumen member may expand into the second body lumen 910 and anchor itself to the second body lumen 910. In such cases, the anchored proximal portion 155 of the stent 150 may maintain the stent 150 in a stationary position as the inner lumen is retracted after the stent 150 fully deploys.

The proximal portion 155 of the stent 150 may include a second flared portion 815. In such cases, the second flared portion 815 may expand within the second body lumen 910 in direct response to retracting the outer sheath 105 past the distal end 930 of the proximal marker 115. As the distal end of the outer sheath 105 is withdrawn past the distal end of the proximal marker 115 and into the isolation sheath 110, the proximal portion 155 of the stent 150 expands to expose the second flared portion 815. As the proximal portion 155 of the stent 150 expands, the second flared portion 815 contacts the wall 925 of the second body lumen 910. The second flared portion 815 (e.g., proximal portion 155) of the stent 150 may be anchored within the second body lumen 910 such that the proximal portion 155 of the stent 150 remains in a fixed position.

Once the stent 150 fully expands, the inner lumen member, the anchoring component 120, the electrocautery tip 125, and the guidewire 145 are withdrawn through the access site 915. Once the distal end of the outer sheath 105 is retracted past the distal end of the proximal marker and into the isolation sheath 110, the inner lumen member, the anchoring component 120, and the electrocautery tip 125 may be removed from the first body lumen 905, through the access site 915, and from the second body lumen 910 after the stent 150 fully deploys.

The first flared portion 810 (e.g., distal portion 160) of the stent 150 may be anchored within the first body lumen 905 such that the distal portion 160 of the stent 150 remains in a fixed position. In such cases, the distal portion 160 of the stent 150 may be compressed against the wall 920 of the first body lumen 905 after deploying the distal portion 160 of the stent 150 from the outer sheath 105. Furthermore, the stent 150 may at least partially cover the access site 915.

The second flared portion 815 (e.g., proximal portion 155) of the stent 150 may be anchored within the second body lumen 910 such that the proximal portion 155 of the stent 150 remains in a fixed position. In such cases, the proximal portion 155 of the stent 150 may be compressed against the wall 925 of the second body lumen 910 after expanding the proximal portion 155 of the stent 150 from within the outer sheath 105. Furthermore, the stent 150 may at least partially cover the access site of the second body lumen 910.

It should be noted that these methods describe possible implementation, and that the operations and the steps may be rearranged or otherwise modified such that other implementations are possible. In some examples, aspects from two or more of the methods may be combined. For example, aspects of each of the methods may include steps or aspects of the other methods, or other steps or techniques described herein.

Referring now to FIG. 10, the inner tubular member 130 is illustrated as inserted through the electrocautery tip 125 and into the tubular sleeve 330 such that a distal end portion of the inner tubular member is overlapped by a portion (e.g., about 6 mm) of the tubular sleeve 330. Also illustrated in FIG. 10 is the coiled electrode wire 405 (FIG. 4) coupled to the electrocautery tip 125. Although the coiled electrode wire 405 is illustrated in FIG. 10, it should be understood that the embodiment of FIG. 10 may be implemented with any one of the coiled electrode wire 405 (FIG. 4), the single electrode wire 505 (FIG. 5), the spiral electrode wire 605 (FIG. 6), or the electrode tube 705 (FIG. 7) without departing from the scope or spirit of the present disclosure.

In certain aspects of the present disclosure, the tubular sleeve 330 may be formed from PTFE and the inner tubular member may be formed from PEEK. Electrosurgical tissue cutting occurs with rapid vaporization of the tissue at a temperature of 100 degrees Celsius or more at a voltage above 200 Volts. High voltage creates electric arcing to connect the air gap between the electrode (e.g., electrode wire 405) and tissue to cause carbonization. Although most tissue cutting occurs around or below 200 degrees Celsius, some studies have shown that the temperature around a cutting electrode can reach about 250 degrees Celsius. However, the temperature near the cutting electrode can reach higher than 250 degrees Celsius due to tissue desiccation, high impedance, procedure duration, etc. Although PEEK and PTFE have similar thermal properties, the mode of deformation due to heating has been observed to be different between the two materials. The melt temperature of PEEK and PTFE is about 340 degrees Celsius. PTFE has a unique structure and, even at the nominal melting point, has considerable strength and can deform plastically to a limited extent. Viscosity of PTFE in the molten state (melt creep viscosity) is so high that high molecular weight PTFE particles do not flow even at temperatures above its melting point. Rather, the particles sinter much like powdered metals, sticking to each other at the contact points and combine into larger particles. PEEK, on the other hand, when subjected to high temperatures (e.g., 340 degrees Celsius or more) deforms and/or melts in such a way that an inner diameter of a PEEK tube tends to narrow and/or occlude. Whereas PTFE, when subjected to high temperatures (e.g., 340 degrees Celsius or more), deforms and/or melts in such a way that the inner diameter of a PTFE tube tends to stay open. Thus, in the specific example of the tubular sleeve 330 being formed from PTFE and the inner tubular member 130 being formed from PEEK, the PTFE tubular sleeve 330 is positioned coaxially over a distal end portion of the PEEK inner tubular member 130 to prevent excessive deformation of the PEEK inner tubular member 130 during electrosurgical cutting (e.g., via the electrode wire 405). In this manner, the PTFE tubular sleeve 330 serves to reduce the amount of heat to which the PEEK inner tubular member 130 is subjected, thereby inhibiting deformation and/or narrowing of the inner diameter of the PEEK inner tubular member 130 such that the inner tubular member 330 maintains sufficient passage of the guidewire 145 (FIGS. 9A and 9B) through the inner tubular member 130. In this example scenario, the distal tip of the PEEK inner tubular member 130 may be disposed about 2mm proximal of the distal edge of the electrode wire 405.

In another aspect of the present disclosure, the tubular sleeve 330 may be formed from silicone. Similarly as described above with respect to the PTFE tubular sleeve 330, the silicone tubular sleeve 330 also serves to reduce the amount of heat to which the PEEK inner tubular member 130 is subjected, thereby inhibiting deformation and/or narrowing of the inner diameter of the PEEK inner tubular member 130 such that the inner tubular member 330 maintains sufficient passage of the guidewire 145 (not shown) through the inner tubular member 130. In aspects of the present disclosure, a distal end portion of the inner tubular member 130 is overlapped by a portion (e.g., about 6 mm) of the tubular sleeve 330.

In aspects of the present disclosure, the tubular sleeve 330 may be formed from a combination of PTFE and silicone. Regardless of whether the tubular sleeve 330 is formed from PTFE, silicone, or both, the tubular sleeve 330 may be a simple single-lumen tube or may, in aspects, be formed to have a stepped section where the inner diameter of the tubular sleeve 330 is expanded at a proximal end portion to accommodate the outer diameter of the inner tubular member 130 within the inner diameter of the tubular sleeve 330 along the overlap.

Although FIG. 10 illustrate the tubular sleeve 330 as flush with the distal edge of the electrode 405, in aspects a distal end of the tubular sleeve 330 may extend distally beyond a distal edge of the electrode 405 (e.g., about 0.25-0.5 mm or about 0.5-3 cm) or may be disposed proximal (e.g., to some desirable distance) to the distal edge of the electrode 405.

Referring now to FIG. 11 in combination with FIG. 4, the cover 415 of the electrocautery tip 410 may include a distal extension 425 that extends from a distal-most end of the cover 415 and that is stepped down relative to the rest of the cover 415. A coiled electrode wire (e.g., coiled electrode wire 405) is looped around the distal extension 425 such that the coiled electrode wire 405 is either stepped down from the outer surface of the rest of the cover 415 or at most flush with the outer surface of the cover 415. A distal end of the extension 425 may be flush with a distal edge of the coiled electrode wire 405 or may extend distally of the distal edge of the coiled electrode wire 405. Although FIG. 11 is illustrated in terms of the distal cutting element 400 described in FIG. 4, it should be understood that the distal cutting elements 500 and 600 described in FIGS. 5 and 6, respectively, may also be implemented to include an electrode wire looped around a distal extension.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed herein.

While several embodiments of the present disclosure have been described and illustrated herein, those of ordinary skill in the art will readily envision a variety of other means or structures for performing the functions or obtaining the results or one or more of the advantages described herein, and each of such variations or modifications is deemed to be within the scope of the present disclosure. More generally, those skilled in the art will readily appreciate that all parameters, dimensions, materials, and configurations described herein are meant to be exemplary and that the actual parameters, dimensions, materials, or configurations will depend upon the specific application or applications for which the teachings of the present disclosure is/are used.

What is claimed is:

1. A system for delivering a stent into a body lumen, comprising:
an inner tubular member configured to advance through an access site in a wall of a body lumen;
a stent configured to be disposed coaxially on the inner tubular member;
an outer sheath disposed coaxially along at least a portion of the inner tubular member such that the stent is disposed between the inner tubular member and the outer sheath while the stent is in an undeployed configuration;
a distal cutting element coupled with a distal end portion of the inner tubular member and having an electrode configured to create the access site in the wall of the body lumen; and
a tubular sleeve disposed coaxially over a distal end portion of the inner tubular member and configured to thermally insulate at least a portion of the inner tubular member.

2. The system according to claim 1, wherein the tubular sleeve is formed of PTFE.

3. The system according to claim 1, wherein the tubular sleeve is formed of silicone, ceramic, or a ceramic-impregnated material.

4. The system according to claim 1, wherein a distal end of the tubular sleeve is disposed distal to a distal edge of the electrode.

5. The system according to claim 1, wherein a distal end of the tubular sleeve is flush with a distal edge of the electrode.

6. The system according to claim 1, wherein a distal end of the tubular sleeve is disposed proximal to a distal edge of the electrode.

7. The system according to claim 1, wherein the tubular sleeve overlaps the distal end portion of the inner tubular member by about 6 mm.

8. The system according to claim 1, wherein a distal end of the inner tubular member is spaced proximally from a distal end of the electrode.

9. The system according to claim 1, further comprising an anchoring component disposed at the distal end portion of the inner tubular member and configured to retain a distal portion of the stent in place along the inner tubular member as the outer sheath is retracted proximally to deploy the stent, wherein upon retraction of the outer sheath, the stent releases from the anchoring component and expands into a deployed configuration within the body lumen.

10. The system according to claim 1, further comprising a proximal marker disposed around the inner tubular member and positioned such that a proximal end of the stent abuts against the proximal marker while the stent is in the undeployed configuration, wherein the proximal marker is configured to indicate a location of the proximal end of the stent.

11. The system according to claim 10, further comprising a middle member disposed around the inner tubular member proximal to the proximal marker such that a proximal end of the proximal marker abuts a distal end of the middle member.

12. The system according to claim 1, wherein the distal cutting element includes a cover having an extension extending distally from a distal end of the cover, and the electrode is looped around the extension.

13. The system according to claim 12, wherein the cover is formed of a material selected from the group consisting of silicone, ceramic, and PTFE.

14. A system for delivering a stent into a body lumen, comprising:
an inner tubular member configured to advance through an access site in a wall of a body lumen for delivering a stent into the body lumen;
an outer sheath disposed coaxially along at least a portion of the inner tubular member;
a distal cutting element coupled with a distal end portion of the inner tubular member and having an electrode configured to create the access site in the wall of the body lumen; and
a tubular sleeve disposed coaxially over the distal end portion of the inner tubular member and configured to thermally insulate at least a portion of the inner tubular member, wherein a distal end of the tubular sleeve is disposed distal to a distal edge of the electrode.

15. The system according to claim 14, wherein the tubular sleeve is formed of PTFE.

16. The system according to claim 14, wherein the tubular sleeve is formed of silicone, ceramic, or a ceramic-impregnated material.

17. The system according to claim 14, wherein the tubular sleeve overlaps the distal end portion of the inner tubular member by about 6 mm.

18. The system according to claim 14, wherein a distal end of the inner tubular member is spaced proximally from a distal end of the electrode.

19. The system according to claim 14, wherein the distal cutting element includes a cover having an extension extending distally from a distal end of the cover, and the electrode is looped around the extension.

20. A system for delivering a stent into a body lumen, comprising:
an inner tubular member formed of PEEK and configured to advance through an access site in a wall of a body lumen for delivering a stent into the body lumen;
an electrode coupled with a distal end portion of the inner tubular member and configured to create the access site in the wall of the body lumen; and
a tubular sleeve formed of PTFE, silicone, ceramic, or a ceramic-impregnated material, the tubular sleeve disposed coaxially over the distal end portion of the inner tubular member and configured to thermally insulate at least a portion of the inner tubular member.
